(19) United States
(12) Patent Application Publication
(10) Pub. No.: US 2007/0161905 A1
(43) Pub. Date: Jul. 12, 2007

(54) INTRAUTERINE ULTRASOUND AND METHOD FOR USE

(75) Inventor: Michael Munrow, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Gynesonics, Inc., Redwood City, CA (US)

(21) Appl. No.: 11/620,569

(22) Filed: Jan. 5, 2007

Related U.S. Application Data
(60) Provisional application No. 60/821,009, filed on Aug. 1, 2006.
(60) Provisional application No. 60/758,727, filed on Jan. 12, 2006.

(51) Int. Cl. A61B 8/14

(52) U.S. Cl. 600/459

(57) ABSTRACT

A method and apparatus for medical imaging is described. The apparatus applies specifically to accessing and targeting tissue in a small cavity or tightly enclosed space. The medical imaging apparatus or device uses ultrasound waves with elements that act as both a transmitter and receiver in order to image body tissues. The ultrasound is an array or plurality of arrays that may be arranged on the tip on a probe or catheter for insertion into a patient's body.

### CROSS-REFERENCES TO RELATED APPLICATIONS

This application is claims priority to U.S. Provisional Application No. 60/758,727 (Attorney Docket No. 025676-000400US), filed on Jan. 12, 2006, and U.S. Provisional Application No. 60/821,009 (Attorney Docket No. 025676-000410US) filed on Aug. 1, 2006, the full disclosures of which are incorporated herein by reference.

### BACKGROUND OF THE INVENTION

The present invention relates generally to medical apparatus and methods. More particularly, the present invention relates to methods and apparatus for ultrasonically imaging fibroids in the uterine cavity.

Ultrasound medical imaging has been known for several decades. Medical ultrasound imaging began using low frequencies (2-5 MHz) for surface imaging of internal body structures. These low frequency approaches generally had good penetration but poor resolution, i.e., ability to see fine images. As technology advanced the ability to make smaller, higher frequency probes became possible. These probes have been used in a variety of imaging procedures over the past several years and have the advantage of great near field resolution. However these probes need to be close to the tissue that they are imaging thus more invasive modalities of imaging have come into practice. Examples are seen in endovaginal, endorectral and transesophageal probes which typically operate in the 5-12 MHz range.

Smaller and higher resolution probes are used in cardiology for imaging of the coronary vasculature as well as the cardiac chambers. These endovascular probes usually operate in the 10-20 MHz range. They often comprise mechanically scanned ultrasound arrays which provide a 360 degree image rather than either a linear or vector type image which most physicians are more comfortable with. While these small, high resolution endovascular probes have also been experimented with in a variety of other tissues and procedures, they remain optimized for intracoronary and intracardiac use.

Miniaturized vector scan phased arrays have recently been introduced for use within the heart and blood vessels. Such ultrasound arrays provide physicians with a clearer, more familiar image format but are generally limited to cardiac use. There have been several studies where investigators have taken a miniaturized side firing phased array transducer mounted to a catheter or a probe and used it for imaging tissues outside the heart. The transducers, however, had not been optimized for use in these tissues.

Gynecologist currently used endovaginal or transabdominal ultrasound to diagnose a variety of diseases relating to women's health. Endovaginal ultrasound has also been used with saline infusion of the endometrial canal to improve imaging of the endometrial tissue. Some researchers have used very high frequency intrauterine sonography with a mechanically rotated transducer in the 10-30 MHz range. This image is limited by the depth of penetration of only a few millimeters and has not found to be clinically useful. Lower frequency endovaginal probes have traditionally been too large (>8 mm diameter) or have had too low a frequency (5-7.5 MHz) to be clinically useful within the uterine cavity.

What is needed is a small ultrasound array that may be inserted directly into the uterine cavity for imaging the endometrium, uterus and surrounding pelvic anatomy for diagnostic and/or therapeutic procedures. More particularly, it would be desirable to provide imaging apparatus and procedures which are capable of detecting fibroids in the uterine wall at varying depths, typically from the surface to depths of 6 cm or greater. Such variable depth imaging should preferably provide high resolutions images which permit accurate interventional treatments with the fibroids that are identified and located. At least some of these objectives will be met by the inventions described hereinafter.

### SUMMARY OF THE INVENTION

The present invention provides improved, small-sized ultrasonic imaging apparatus intended for transcervical introduction into the uterus for imaging of the uterine wall. The apparatus and methods of the present invention will be particularly suitable for imaging fibroids disposed at virtually any depth within the uterine wall, typically being anywhere between the surface of the uterine wall to a depth of 6 cm or more. Advantageously, the present invention further provides for adjusting the imaging penetration of the ultrasonic array so that good resolution of the fibroids or other uterine structures can be obtained over the entire range of depths from 0-6 cm or more within the uterine wall. Typically, the imaging penetration is varied by changing the operational frequency of that transducer, typically over the range from 5 MHz to 12 MHz.

In a first aspect of the present invention, an ultrasound probe assembly comprises a probe body adapted to access a uterus or other body cavity in an ultrasonic imaging transducer array disposed on or in a distal region of the probe. The array will be a phased array, usually including at least 32 elements, with an azimuthal aperture of at least 5 mm. Typically, the array will include at least 64 elements, with a linear pitch in at least 13 mm of azimuthal aperture, often having 12 mm of azimuthal aperture or more. Potentially, the array will include at least 128 elements, with a linear pitch of azimuthal aperture of 15 mm or more. The ultrasonic imaging transducer array will typically operate at a frequency in the range from 5 MHz to 12 MHz, more typically being adjustable within that range to provide for an adjustable imaging penetration. The adjustable imaging penetration will typically include at least two depths within the range from 0.1 cm to 8 cm within the uterine wall, typically being from 0.5 cm to 5 cm. Optionally, a distal region of the probe may be deflectable or inclined relative to a proximal portion of the body in order to facilitate scanning and imaging of the uterine wall. Alternatively, the ultrasonic imaging transducer could be removably positioned within the probe body so that the transducer could be reused while the body is disposable. See copending application Ser. No. 11/564,164 (Attorney Docket No. 025676-000720US), the full disclosure of which is incorporated herein by reference. Optionally, the array may be rotatable about the long axis of the device to facilitate scanning in the elevational direction. Alternately, the probe may include another linear set of elements, orthogonal to the first set, which constitute a biplane transducer.

In a further aspect of the present invention, methods for imaging uterine fibroids in a uterine wall comprise advancing an ultrasonic imaging transducer array into a uterine cavity. A region of the uterine wall is imaged with the ultrasonic imaging transducer array, where the transducer array is operated with an imaging penetration in a range from 0.1 cm to 8 cm within the wall. The same or another region of the uterine wall is then imaged with the same transducer array, where the transducer array is operated with a second imaging penetration in a range from 0.1 to 8 cm within the wall. Successive regions and/or depths within the wall may then be successively scanned in order to identify fibroids within the wall as well as to determine the dimensions of such fibroids in order to assist in treatment. Typically, the imaging penetration will be changed by changing the frequency of operation of the transducer array, usually within a range from 5 MHz to 12 MHz.

The methods of the present invention may further comprise treating any or all of the uterine fibroids which have been identified. Treating may comprise advancing a treatment tool into or adjacent to the identified uterine fibroid, typically while continuing to image the fibroid to make sure the treatment tool is properly oriented. The treatments typically comprise advancing a needle to engage or penetrate the uterine wall at or near the uterine fibroid, where treatment energy and/or a treatment agent is delivered by the needle into the fibroid, as described in detail in copending application Ser. No. 11/409,496 (Attorney Docket No. 025676-000700US), the full disclosure of which is incorporated herein by reference.

### BRIEF DESCRIPTION OF THE DRAWINGS

FIG. 1 illustrates an ultrasound probe or catheter constructed in accordance with the principles of the present invention.

FIG. 1A is a detailed end of the distal view of the ultrasound probe or catheter of FIG. 1, showing the phased array ultrasound transducer.

FIG. 2 illustrates a reusable probe or catheter constructed in accordance with the principles of the present invention having a sterile ultrasound drape.

FIG. 3 illustrates an ultrasound probe or catheter without an attached handle.

FIGS. 4A-4C illustrate use of the ultrasound probe or catheter of the present invention for imaging and treating uterine fibroids in a uterine wall, where the fibroids are at different depths.

### DETAILED DESCRIPTION OF THE INVENTION

The present invention provides a very small diameter probe or catheter for access to the interior of the uterus with little or no dilatation of the cervix, typically having a width or diameter from 2 mm to 10 mm, usually from 3 mm to 8 mm. The exemplary probe includes a 64 element phased ultrasonic array with a 13 mm aperture, although as few as 32 elements or as many as 128 elements may be used as well. The aperture of the array may also be in the range from 6 mm to 14 mm. Increasing the aperture size is advantageous since the resolution of the image is improved. Electronic steering of the ultrasound beams (±90°, usually ±45° depending on the frequency of operation and the ultrasound element spacing) may also be provided, with the frequency of operation from 5 to 12 MHz. Depending on the target that is being imaged the frequency may be changed to change resolution and imaging penetration. For example, to image the endometrial cavity one may use a higher frequency and then switch to a lower frequency to image large myomas. The elevation aperture will typically be in the range from 1 mm to 6 mm, usually being 2.5 mm, and the imaging depth is optimal from 0.5 cm to 6 cm in order to easily see uterine, fallopian and ovarian pathology as well as anatomically close extrauterine organs such as the bladder or the bowel. This elevation aperture may be increased to improve the slice thickness of the ultrasound beam. A lens may be used in front of the array to focus the ultrasound energy in either or both the elevation and/or azimuthal directions.

The devices of the present invention typically comprise probes or other elongated instruments which are suitable for transvaginal, transcervical and intrauterine scanning, wherein the probes carry ultrasonic transducer arrays capable of operating in the B mode, Color Doppler, Power Color Doppler, PW Doppler, and the like. Advantages over conventional endovaginal or transabdominal imaging include a closer and/or higher resolution view of the anatomy that may allow diagnosis of previously indistinct pathology as well as a platform from which to perform therapeutic ultrasound guided procedures. The probe or catheter may have mechanical steering and/or rotation of the tip to allow better access to anatomy as needed. In addition the probe or catheter may have a working channel for infusion and replenishment of ultrasound coupling medium (gel, water, etc.), and may further comprise an electrode or other interventional tool for treating the fibroid or other tissue structure. Alternatively, infusion of materials and/or introduction of tools may be performed through the lumen of a separate introducing tool as taught, for example, in copending provisional application No. 11/564,164 (Attorney Docket No. 025676-000300US), previously incorporated herein by reference.

The imaging probe is usually connected to a dedicated gynecology specific ultrasound console using a cable or other connector, and said console may have the ability to stitch images together to get a panoramic image (extended field of view). It is also possible to have three dimensional ultrasound capability for the probe and the system in order to obtain a three dimensional view of the entire uterus and surrounding tissue.

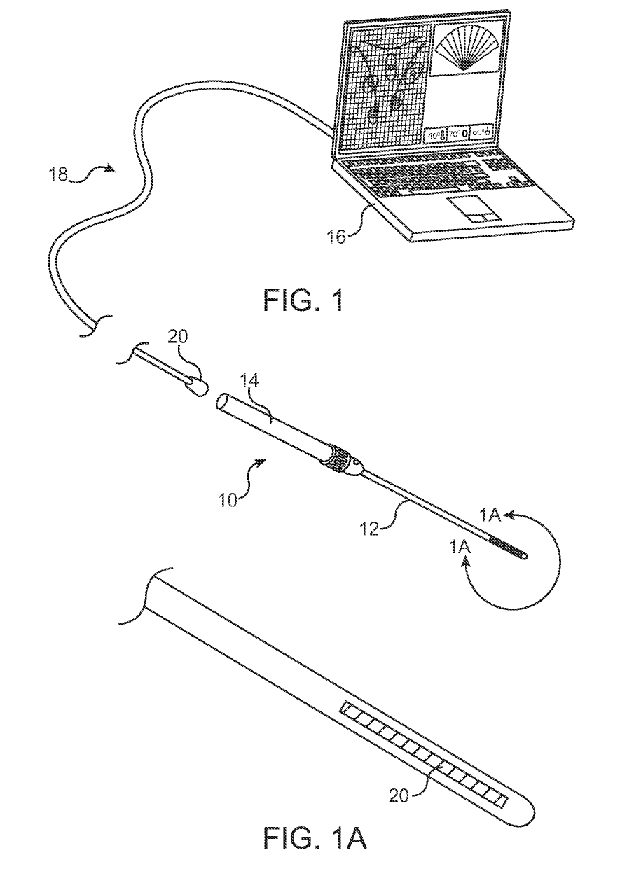

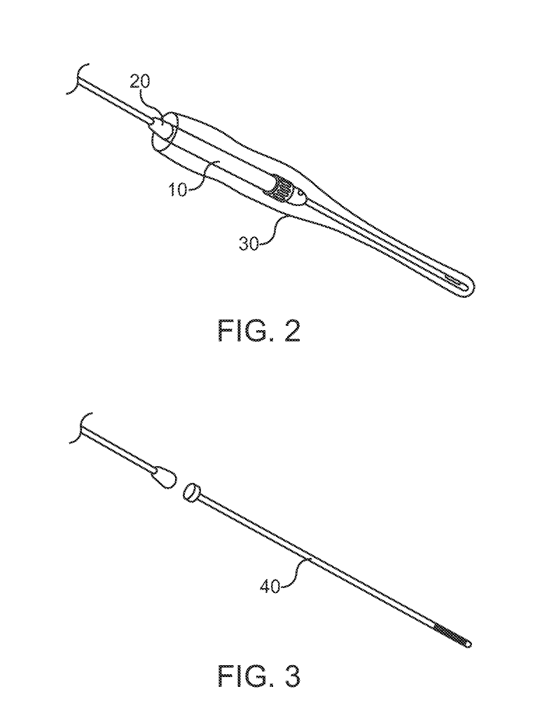

As shown in FIGS. 1 and 1A, a probe 10 comprises a shaft 12 having a handle 14 for manipulation that is connected to a portable imaging engine 16 (a laptop computer programmed with imaging software) by a cable 18. An intrauterine image is shown on the console screen. The shaft 12 of probe 10 is small enough so that it may easily be inserted into a patient's vagina and through her cervix with minimal pain or dilatation. In this embodiment the device is a sterile, single use device. The cable 18 may comprise a conventional coaxial cable, where the connection to the ultrasound array 20 (FIG. 1A) through the shaft 12 and handle 10 is provided by flex circuits running through the device. Alternatively, the flex circuit may extend through the entire length of the cable from the ultrasound array 20 to the portable imaging engine 16 to provide the connection. A connector 20 at the end of cable 18 will be provided with appropriate connectors for interfacing between the flex circuitry and the coaxial cable.

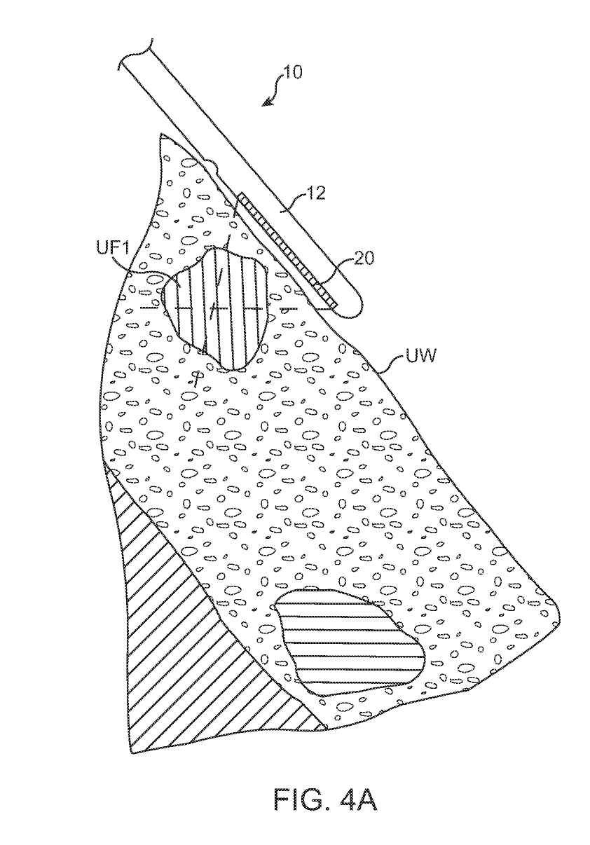

Referring to FIG. 2, the probe or catheter 10 may be inserted into a sterile ultrasound drape 30. The device and the drape may be used with ultrasound coupling gel or fluid. In this embodiment the device is reusable.

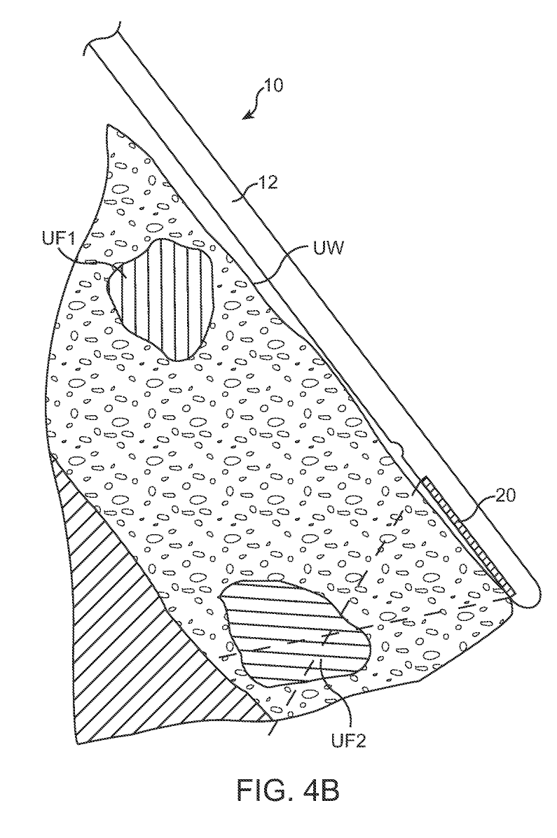

FIG. 3 illustrates an ultrasound core 40 with little to no handle attached. The ultrasound core will typically be provided with an external device with which to hold and manipulate the ultrasound core, as taught, for example, in copending provisional application No. 60/758,881, the full disclosure of which has been incorporated by reference. The two devices may inserted together into the uterus, then anatomy can be visualized by a number of logical scanning sequences. One such scanning sequence is to start visualizing and recording from the 12 o'clock position, proceeding clockwise from the fundus, retracting 1 cm at each full rotation of the clock. The portable ultrasound engine provides the ability to capture, record and store images. Color Doppler, Power Doppler, Power Color Doppler, PW Doppler, or B mode may optionally be used. The device combination may then be removed and reused and/or disposed of. Images and clips which are captured may be printed, archived to removable digital storage media, or sent over a network for storage and/or image manipulation.

Exemplary ultrasound transducer arrays 20 may be obtained from commercial sources. A first exemplary ultrasound array will have 64 elements, with an 0.110 mm pitch, with a 7 mm aperture (Azumith), available from Tetrad Corporation, Englewood, Colo. as Model No. TC-800-CATH. A second exemplary ultrasound array has 64 elements with an 0.205 mm pitch, and a 13 mm aperture (Azumith), available from Vermon, Tours, France, under the tradename Gastro.

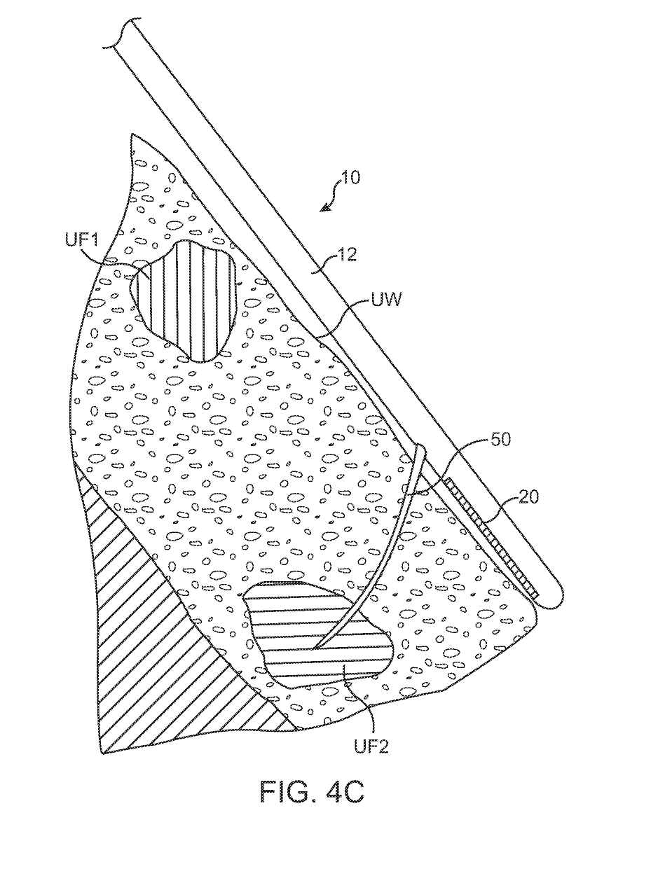

Referring now to FIGS. 4A-4C, a probe or catheter 10 may be introduced transvaginally into a uterine cavity so that the ultrasound array 20 is engaged against the uterine wall. Typically, the probe may be generally rigid, steerable, deflectable, or the like, or present in a rigid carrier, sheath or other external support structure. Alternatively, the probe may be non-rigid. A particular probe design employing a non-rigid imaging core removably disposed in a rigid shaft or sheath is described in copending application Ser. No. 11/564,164 (Attorney Docket No. 025676-000710US), the full disclosure of which is incorporated herein by reference. As shown in FIG. 4A, the ultrasound transducer array 20 is positioned over a first uterine fibroid UF1 which may be imaged, typically by controlling the imaging penetration so that a high resolution image of the fibroid may be obtained. Conveniently, the imaging penetration may be changed by adjusting the operational frequency of the array.

The catheter 10 can also be used in a scanning mode when the uterus is filled with a sound conductive fluid and the imaging array back away from the wall region being scanned. Regions which appear to have a fibroid (based on observed echogenicity, distortion, and posterior shadowing) may then be imaged more closely by advancing the transducer array against the wall surface above the suspected fibroid. This technique is also useful for detailed imaging of submucosal fibroids which are located at the surface of the uterine wall.

After locating the first uterine fibroid UF1, the catheter of probe 10 may be advanced until the ultrasonic array 20 locates a second uterine fibroid UF2 which is located at a greater depth in the uterine wall than the first fibroid. After locating the second uterine fibroid UF2, the imaging penetration of the transducer array 20 may be adjusted to provide for a high resolution image of the array.

When imaging either the first or second uterine fibroid UF1 or UF2, treatment of the uterine fibroid may be effected using an interventional tool on the catheter or probe 10, or alternatively on a sheath, shaft, or other delivery or placement device as described in copending application Ser. No. 11/564,164, the full disclosure of which has previously been incorporated herein by reference. For example, as shown in FIG. 4C, a needle 50 may be advanced from a side port of the shaft 12 and introduced into the second uterine fibroid UF2, typically while the fibroid is being imaged in real time. Thus, the physician can make sure that the needle has penetrated the uterine fibroid at a desired location and to a desired depth. Once the needle is properly placed, it can be used to deliver radiofrequency energy to treat the uterine fibroid, as described in copending application Ser. No. 11/409,496 (Attorney Docket No. 025676-000700US).

Alternatively, the needle or other structure could be used to deliver energy into the pericapsular region (surrounding the uterine fibroid), as described in provisional application No. 60/821,006 (Attorney Docket No. 025676-001000US), filed Aug. 1, 2006.

While the above is a complete description of the preferred embodiments of the invention, various alternatives, modifications, and equivalents may be used. Therefore, the above description should not be taken as limiting the scope of the invention which is defined by the appended claims.

What is claimed is:

1. An ultrasound probe assembly comprising:
a probe body adapted to access a body cavity; and
an ultrasonic imaging transducer array disposed on or in a distal region of the probe, said array comprising at least 32 linear elements with an azimuthal aperture of at least 5 mm.

2. An ultrasound probe as in claim 1, wherein the array includes at least 64 elements with a linear pitch with at least 7 mm of azimuthal aperture.

3. An ultrasound probe as in claim 1, wherein the array includes at least 64 elements with a linear pitch with at least 12 mm of azimuthal aperture.

4. An ultrasound probe as in claim 1, wherein the array includes at least 128 elements with a linear pitch with at least 15 mm of azimuthal aperture.

5. An ultrasound probe assembly as in claim 1, wherein the probe body is adapted for transcervical introduction in a uterus.

6. An ultrasound probe assembly as in clam 1, wherein the ultrasonic imaging transducer array operates at a frequency in the range from 5 MHz to 12 MHz.

7. An ultrasound probe assembly as in claim 1, wherein the operation of the ultrasonic imaging transducer array can be switched between at least two different frequencies to control imaging depth.

8. An ultrasound probe assembly as in claim 7, wherein the imaging depth can be adjusted to at least two depths in the range from 0.1 mm to 8 mm.

9. An ultrasound probe assembly as in claim 1, wherein the ultrasonic imaging transducer array has an elevation aperture in the range from 0.5 cm to 5 cm.

10. An ultrasound probe assembly as in claim 1, wherein the distal region of the probe is deflectable.

11. An ultrasound probe assembly as in claim 1, wherein the distal end of the probe has a fixed deflection.

12. An ultrasound probe assembly as in claim 1, wherein the ultrasound imaging transducer array is adapted to provide B mode, C mode, Color Doppler, PW Doppler, and/or Power Color Doppler scanning.

13. An ultrasound probe assembly as in claim 1, wherein the ultrasonic imaging transducer is removably receivable in the probe body.

14. An ultrasound probe assembly as in claim 1, wherein the ultrasonic imaging array comprises a biplane transducer having a second linear set of elements orgothonal to the first linear set of elements.

15. An ultrasound probe assembly as in claim 1, wherein the transducer array is rotatable about a central axis of the probe body.

16. A method for imaging uterine fibroids in a uterine well, said method comprising:
advancing an ultrasonic imaging transducer array into a uterine cavity;
imaging a region of the uterine wall with the ultrasonic imaging transducer array, wherein the transducer array is operable with an imaging penetration in the range from 0.1 cm to 8 cm in the wall; and
imaging a region of the uterine wall with the ultrasonic imaging transducer array, wherein the transducer array is operated with a second imaging penetration in the range from 0.1 cm to 8 cm in the wall.

17. A method as in claim 16, wherein the imaging penetration is changed by changing the frequency of operation of the transducer array within a range from 5 MHz to 12 MHz.

18. A method as in claim 16, wherein the imaging transducer array comprises at least 32 elements with an azimuthal aperture of at least 5 mm.

19. A method as in claim 16, wherein at least one uterine fibroid is located in at least one of the imaging steps.

20. A method as in claim 19, further comprising treating said at least one uterine fibroid.

21. A method as in claim 20, wherein treating comprises advancing a treatment tool into or adjacent to the at least one uterine fibroid while continuing to image said fibroid.

22. A method as in claim 21, wherein treating comprises advancing a needle to penetrate the uterine wall at or near the uterine fibroid, wherein treatment energy is delivered to the fibroid through the needle.

23. A method as in claim 22, wherein the treatment energy is selected from the group consisting of radiofrequency, microwave, high intensity focused ultrasound, and cryotherapy.
